(19) United States
(12) Patent Application Publication
(10) Pub. No.: US 2007/0031500 A1
(43) Pub. Date: Feb. 8, 2007

(54) Long-acting solid formulation comprising triptorelin acetate

(75) Inventors: Roland Cherif-Cheikh, Castelldefels (Barcelona) (ES); Francesc Navarro Pujol, Barcelona (ES)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Societe de Conseils de Recherches et D'Applications Scientifiques, Paris (FR)

(21) Appl. No.: 11/499,777

(22) Filed: Aug. 7, 2006

(30) Foreign Application Priority Data
Feb. 5, 2005 (FR) 0401109

(51) Int. Cl. A61K 9/14; A61K 31/4439

(52) U.S. Cl. 424/486; 514/343

(57) ABSTRACT

The invention relates to a long-acting solid formulation for parenteral administration, comprising a) triptorelin acetate and b) one or more excipients comprising a polymer or copolymer of lactic and/or glycolic acid or a mixture of polymers and/or copolymers of lactic acid and/or glycolic acid, said formulation containing 10 to 99% of triptorelin acetate by weight with relation to the total weight of the formulation. Said formulation is obtained by means of a method comprising the fusion of a mixture of triptorelin acetate and the excipient(s) on the fusion/extrusion of the triptorelin acetate with the excipient(s), said formulation being embodied such as to release the triptorelin acetate over a duration of at least one week, once administered parenterally to a patient.

### FIELD OF INVENTION

A subject of the present invention is a solid sustained-release formulation for parenteral administration comprising triptorelin acetate.

### BACKGROUND OF INVENTION

Triptorelin (also known by the name [D-Trp⁶] LHRH) is an analogue of the hormone LHRH. This decapeptide intended to treat, in particular, prostate cancer or endometriosis, is currently used as the active ingredient in the medicament Decapeptyl® (also called Diphereline® in certain countries).

The Applicant had already described, in the PCT Patent Application WO 98/24504, a solid sustained-release formulation for parenteral administration comprising a homogeneous mixture of an active ingredient (in particular a triptorelin salt) in a dispersed or non-dispersed state forming a continuous phase at least part of which is in direct contact with the exchange surface of the formulation and the exterior biological medium, and a biodegradable biocompatible excipient (in particular a lactic and/or glycolic acid polymer or copolymer or a mixture of lactic and/or glycolic acid polymers and/or copolymers), in which the quantity of active ingredient is at least 50% by weight with respect to the total weight of the formulation, and having a release profile independent of the composition of the excipient, the molecular weight of the excipient or the active ingredient/excipient weight ratio, the release profile being essentially exclusively dependent on the total quantity of active ingredient present in the formulation.

The Applicant has now discovered that the properties of such sustained-release formulations could be further improved for triptorelin acetate. In particular, the Applicant has developed sustained-release triptorelin acetate formulations the initial release burst of which is also reduced relative to the standard formulations such as those described in the PCT Patent Application WO 98/24504.

The Applicant has also discovered production conditions, according to an advantageous process, making it possible to obtain certain said sustained-release formulations.

In fact, a formulation according to the invention does not have an isolated initial release burst but rather a maximum release at the start which stabilizes regularly towards the necessary and sufficient sustained-release profile (circulating level). The continuity in the dose of triptorelin delivered represents an important advantage of this type of formulation as the dose circulating in the patient can thus be maintained at sufficient levels in order to obtain a therapeutic effect and the circulating triptorelin concentration will remain, thanks to injections repeated at regular intervals and with a release profile without an initial burst and without troughs, greater than or equal to the requirements of the treatment. The Applicant has thus discovered that the use of formulations having these release profile characteristics made it possible to increase the treatment intervals and to reduce the total dose with lower circulating concentrations of active ingredient than those used to date; thus getting closer to the minimum therapeutic doses, which allows considerable economies in active ingredient and therefore formulation for the same treatment.

### BRIEF SUMMARY OF THE INVENTION

A subject of the invention is therefore a solid sustained-release formulation for parenteral administration comprising:

a) triptorelin acetate, and

b) one or more excipients comprising a lactic acid and/or glycolic acid polymer or copolymer or a mixture of lactic acid and/or glycolic acid polymers and/or copolymers,

said formulation containing 10 to 99% triptorelin acetate by weight relative to the total weight of the formulation and being obtained by a process comprising the melting of the mixture of triptorelin acetate and excipient or excipients during the melting-extrusion of the triptorelin acetate with the excipient or excipients, said formulation being such that it releases the triptorelin acetate over a period of at least one week once administered to a patient by parenteral route.

Preferably, a formulation according to the invention, once administered to a patient by parenteral route, will release the triptorelin acetate at an effective dose over a period of at least 14 days (more preferentially over a period of at least 28 or 30 days, and still more preferentially over a period of at least 60, 90, 120 or even 180 or 360 days).

### DETAILED DESCRIPTION OF THE INVENTION

According to the invention, the solid sustained-release formulation will preferably comprise 20 to 90%, more preferentially 25 to 80% and still more preferentially 30 to 70% by weight of triptorelin acetate relative to the total weight of the formulation. In particular, the solid sustained-release formulation of the invention can comprise 35 to 55% by weight of triptorelin acetate relative to the total weight of the formulation.

Preferably, the melting of the mixture of the triptorelin acetate and the excipient or excipients takes place at the same time as the extrusion of said mixture leading to the sustained-release formulation of the invention.

Preferably, when it comprises more than 35% triptorelin acetate by weight relative to the total weight of the formulation, said formulation is such that it releases in less than one week (and preferably in less than 48 hours) virtually all of the triptorelin acetate which it contains in 500 ml of an aqueous solution of pH 6.0 containing 0.9% by weight of sodium chloride and maintained under stirring at a speed of 25 rpm at a temperature comprised between 25 and 37° C., preferably between 30 and 37° C. and particularly at approximately 30° C., but also such that it releases the triptorelin acetate over a period of at least one week once administered to a patient by parenteral route, and is moreover characterized in that the quantity of residual water in the mixture of triptorelin salt with excipients incorporated into said sustained-release formulation will not exceed 8% by weight of water relative to the total weight of said mixture.

By triptorelin acetate, unless otherwise specified, is meant in the present Application more than 95% by weight pure triptorelin acetate, and preferably more than 97 or 98% pure expressed by weight of triptorelin acetate. This corresponds respectively to a percentage of the order of approximately 80, 84 or 85% by weight of peptide.

By virtually all of the triptorelin acetate is meant more than 80% of the initial quantity of triptorelin acetate, and more preferentially more than 90 or even 95% of this quantity.

According to a variant of the invention, the quantity of triptorelin acetate is at least 55% or even 60% by weight relative to the total weight of the formulation, and more preferentially at least 70% or even 75% by weight relative to the total weight of the formulation. Moreover, still according to this variant of the invention, the quantity of lactic acid and/or glycolic acid polymer or copolymer or a mixture of lactic acid and/or glycolic acid polymers and/or copolymers is preferably at least 20% by weight relative to the total weight of the formulation, and more preferentially at least 25% or even 30% by weight relative to the total weight of the formulation.

According to another variant of the invention, the quantity of triptorelin acetate is 35 to 55% (and more preferentially 35 to 50%) by weight relative to the total weight of the formulation.

According to the invention, the lactic acid and/or glycolic acid polymer or copolymer or the mixture of lactic acid and/or glycolic acid polymers and/or copolymers is preferably a lactic acid and glycolic acid copolymer or a mixture of such copolymers.

All the types of lactic acid and glycolic acid (PLGA) copolymers can be used for the compositions according to the invention, and in particular a 50-50 PLGA (i.e. a lactic acid and glycolic acid (PLGA) copolymer comprising 50% units derived from lactic acid and 50% units derived from glycolic acid), a 75-25 PLGA (i.e. a lactic acid and glycolic acid (PLGA) copolymer comprising 75% units derived from lactic acid and 25% units derived from glycolic acid), an 80-20 PLGA (i.e. a lactic acid and glycolic acid (PLGA) copolymer comprising 80% units derived from lactic acid and 20% units derived from glycolic acid) or also an 85-15 PLGA (i.e. a lactic acid and glycolic acid (PLGA) copolymer comprising 85% units derived from lactic acid and 15% units derived from glycolic acid). Generally, it is preferable to use for the solid sustained-release formulations of the invention PLGAs comprising 50% to 85% units derived from lactic acid and 15% to 50% units derived from glycolic acid, in particular PLGAs comprising 70% to 85% units derived from lactic acid and 15% to 30% units derived from glycolic acid, and in particular a PLGA comprising approximately 75% units derived from lactic acid and approximately 25% units derived from glycolic acid (i.e. an approximately 75-25 PLGA). Said PLGAs will possess a more or less short chain as a function of the period of release of the active ingredient sought. Moreover, it is possible to use pure lactic acid (PLA) polymers, in particular for the forms intended to obtain a release over a period of more than 3 months.

Said polymers or copolymers are preferentially used in a form which is purified or devoid of the residual monomer fraction. Polymers or copolymers of this type are for example described in U.S. Pat. No. 4,728,721.

When the lactic acid and/or glycolic acid polymer or copolymer or the mixture of lactic acid and/or glycolic acid polymers and/or copolymers comprises a PLGA, the latter will preferably have a molecular mass of at least 60,000 g/mol, more preferentially at least 75,000 or even 90,000 or 95,000 g/mol (and in particular approximately 100,000 g/mol). When the lactic acid and/or glycolic acid polymer or copolymer or the mixture of lactic acid and/or glycolic acid polymers and/or copolymers comprises a PLA, the latter will preferably have a molecular mass comprised between 15,000 or 20,000 and 30,000 or 40,000 g/mol (in particular approximately 25,000 g/mol).

The sustained-release formulations according to the invention allow the use of a wide variety of polymers with in particular favourable results before or after radiosterilization despite the change in molecular weight, therefore allowing for example an aseptic or gamma-irradiated preparation.

As a function of the polymer or copolymer used and its molecular weight, it can be useful to add a small percentage of PLGA of low molecular weight (2,000 to 6,000 g/mol for example) in order to reduce the extrusion temperature, increase the plasticity or hydrophilic character capable of encouraging the rapid sustained-release control by rearrangement. This small percentage is preferably comprised between 0 and 5%, more preferentially between 0 and 2% and more preferentially between 0 and 1%.

Preferably, still according to the variant of the invention according to which the formulation comprises more than 50% of triptorelin acetate by weight relative to the total weight of the formulation, the mixture of triptorelin acetate with the polymer or copolymer excipient or excipients is previously dried so that its water content does not exceed 8% by weight (preferably 4 or 5% and quite particularly 2%).

The preferred production process according to the invention comprises the mixing under dry conditions of said triptorelin salt in proportions which can range up to more than 50% with said polymer or copolymer excipient or excipients. Said mixture is then compacted and granulated under dry conditions at a temperature below or equal to 25° C. The mixture is then dried in order to have a residual moisture which does not exceed 8% and is preferentially below 4 or 5% or also approximately equal to 2%. Said mixture is then taken to its melting temperature directly and rapidly during the extrusion process.

The mixture of triptorelin acetate with the lactic acid and/or glycolic acid polymer or copolymer or the mixture of lactic acid and/or glycolic acid polymers and/or copolymers is then in the molten state.

Said mixture is thus fed into an extrusion screw according to a process such that the time taken by liquefaction-melting of the mixture and transit up to the extrusion nozzle is reduced and is less than 30 minutes and preferentially less than 15 minutes.

According to this advantageous variant of the production process, the operation is carried out without pretreatment of the mixture using aqueous or organic solvents and/or without lyophilization of the mixtures and without distinct preheating for compression before extrusion, which makes it possible to control, if appropriate, the low hydration state of said mixture and to extrude at temperatures which can be above 100° C. without degradation of the active ingredient, over short heating periods of less than 15 minutes, preferably comprised between 5 and 10 minutes.

This production process avoids the use of production solvent or vehicle to be eliminated subsequently. The solid mixture of triptorelin acetate powder and lactic acid and/or glycolic acid polymer(s) or copolymer(s) can be melted at a sufficient temperature in order to obtain a non-solid state of the two constituents to then be mixed and then extruded or moulded before lowering the temperature and returning the arrangement to the solid state.

In particular, when the excipient is a lactic acid and glycolic acid (PLGA) copolymer comprising approximately 75 to 85% units derived from lactic acid and approximately 15 to 25% units derived from glycolic acid (i.e. an approximately 75-25 or approximately 85-15 PLGA) of approximately 1.1 to 1.6 dl/g viscosity measured in hexafluoroisopropanol (HFIP), the triptorelin acetate should preferably be shaped at temperatures comprised between 110 and 160° C., and more preferentially between 125° C. and 150° C. or even between 137 and 145° C., for example at approximately 143° C.

It is very important to note that at this temperature the peptide is molten which was not the case with the known processes of the prior art and in particular that described in the French patent 2,650,182 (Debiopharm). The fact of being able to operate at a temperature at which the peptide is molten is unexpected as it could have been feared that the peptide would degrade at this temperature which is not the case. This molten or liquefied state of the peptide in the polymer is what allows its mixing without having recourse, as in the state of the art, to expensive pretreatments using production vehicles which must be eliminated subsequently.

This temperature can of course be adapted as a function of the polymer or copolymer used; it will for example be approximately 10° C. lower in the case of an approximately 50:50 PLGA or approximately 10° C. higher for a 75-25 PLGA with a higher viscosity.

As regards the residual moisture in the mixture of triptorelin salt and polymer(s) and/or copolymer(s) incorporated into said sustained-release formulation, the latter is preferably less than or equal to 4 or 5% (more preferentially less than or equal to approximately 2%) by weight of water relative to the total weight. In particular, it is comprised between 1.5 and 2.5% by weight of water relative to the total weight, and quite preferentially between 1.8 and 2.2% by weight of water relative to the total weight (for example approximately 2% by weight of water relative to the total weight).

The Applicant has noted that such proportions for the residual moisture make it possible to obtain advantageous results, in particular as regards the mixture in the molten state and to obtain a release without initial burst and according to the therapeutic doses sought over time. This reduction of the release burst also leads to an extension of the release period relative to a given quantity of triptorelin salt incorporated into the sustained-release formulation. In this manner, it is possible to obtain a release of the triptorelin salt over a period of more than 15, 30, 60, 90 or even 120 or 180 days whilst using (relatively) small quantities of triptorelin salt. Thus, the sustained-release formulations according to the invention will comparatively have a volume smaller still than previously, which will reduce the discomfort experienced by the patient during their injection.

It is thus possible according to the invention to produce triptorelin acetate formulations used for prostate cancer no longer having, as a function of the period of action, an average monthly dose of 3 mg but for example an average monthly dose of 2.5 mg or even 2 mg or also 1 mg. The invention therefore also relates more generally to triptorelin acetate formulations comprising a polymer excipient (in particular a PLGA) or a mixture of polymer excipients, said formulations being capable of releasing, at a dose which is effective in the treatment of prostate cancer and over a period of at least one month, triptorelin acetate into the organism of the patients to whom they are administered, said formulations containing 1 to 2 mg (and in particular approximately 1.5 mg) of triptorelin acetate per month released at an effective dose of triptorelin acetate.

For example in a formulation according to the invention, monthly formulations could contain of the order of 1.5 mg of active ingredient such as triptorelin acetate, compositions envisaged for 4 months' release could contain of the order of 1.5 mg/month of active ingredient (i.e. approximately 6 mg), and compositions envisaged for a treatment period of 6 months could contain of the order of 1.5 to 2 mg/month (i.e. approximately 9 to 12 mg).

In order to preserve these conditions, it may be necessary to manipulate the powder, either in an (enclosed) controlled environment (under dry air or nitrogen flow), or under a heat source maintaining or reducing the ambient humidity (visible light or IR).

In the case of extrusion, this arrangement at a high temperature can produce the desired form directly thanks to the screw mixture and the diameter of the extrusion nozzle.

It is also possible to carry out monitoring of the solid form and in particular of its diameter thanks to an extrusion machine regulating the diameter of the extrudate.

In this case and according to the desired diameter, the extrusion machine can operate at ambient temperature at the extruder outlet. The extrudate can also pass through a thermostatically-controlled chamber at a high temperature, equal to or below the extrusion temperature in order to allow greater extrusion and in particular the obtaining of very small diameters (for example less than 0.1 mm or also less than 0.05 mm).

This continuous extrudate can then be cut to size (exchange surface) offering the desired release profile, for example by cryogrinding. The desired dose can be obtained and injected in the form of one or more pellets or in the form of a microgranulated and calibrated powder.

Depending on the form, dose and desired release profile, this production process can also be applied to forms with small loadings of active ingredient, less than 20%, in particular comprised between 0.5 and 10% or high loadings, greater than 50% and in particular comprised between 60 and 80%.

The indications given above in terms of residual moisture and quantity of active ingredient as well as of the nature of the polymer for example, can be applied to compositions having loadings of less than 50% as well as those having loadings greater than this value. The adaptations necessary are within the scope of a person skilled in the art considering the indications given above as well as in the production examples.

These can therefore be one or more solid forms with a length which can be greater than 1 cm or less than 0.1 mm, according to the case and injected either as an implant, or in the form of a suspension.

In order to obtain dispersed forms, extruded at a high temperature and with a small diameter, it is also possible to use an extrusion nozzle with several channels allowing the output in parallel of several extrudates from the same screw. These extruded threads with a small diameter (less than 0.1 mm) can be cut mechanically to regular lengths (for example 0.05 mm) or also cryo-ground at a low temperature (liquid nitrogen) according to fracture points in order to obtain dispersed forms.

Apart from these techniques which play on the temperature, it is possible to obtain such dispersed forms using solutions by taking advantage of the considerable solubility of the triptorelin salt and small quantity of polymer necessitated by the sustained-release formulations according to the invention, to the extent that the monthly dose is reduced and/or the loading of active ingredient is increased.

These solutions can be prepared in organic solvents miscible with water (for example in acetic acid) or also in supercritical fluids (for example in CO₂ in the supercritical state). These mixtures in solution are then dried or lyophilized then treated in extrusion or directly nebulized, optionally under pressure.

As regards the preparation process of a microimplant according to the invention, if it has been mentioned previously that the preferred process comprised the mixing under dry conditions of the triptorelin salt with said polymer or copolymer excipient or excipients before compacting and granulating under dry conditions, satisfactory alternative methods will consist of omitting the compacting or carrying out this compacting by means of a first extrusion before using the mixture obtained for an extrusion under the temperature conditions described previously.

According to a preferred variant of the invention, the solid sustained-release formulation is presented in the form of a microimplant, i.e. a cylinder with a small diameter (less than 1.5, 1, 0.8, 0.6, 0.5, 0.25 or even 0.1 mm) and a few mm in length, said length preferably being comprised between 5 and 50 mm (more preferentially between 10 and 30 or 40 mm). Preferably, the cylinder will have a length/diameter ratio at least equal to 10, and more preferentially at least equal to 12, or even at least equal to 15 or 20.

In particular:

When the excipient used is an approximately 75-25 PLGA with a molecular mass of approximately 100,000 g/mol, the microimplant according to the invention can have, for example, a diameter of 0.8 to 0.9 mm and comprise 70% by weight of triptorelin salt and 30% by weight of approximately 75-25 PLGA. In such a case, the microimplant according to the invention will have a weight proportional to the period of release sought, i.e. approximately 4.6 to 5.6 mg for a period of release of approximately 1 month or approximately 13.7 to 16.7 mg for a period of release of approximately 3 months. It is also possible here to use the release profile according to the invention in order to produce microimplants of only 15.2 to 18.5 mg for a period of release of approximately 4 months or also microimplants of 18.2 to 22.2 mg for a period of release of approximately 6 months or even smaller microimplants corresponding to average monthly doses of triptorelin of less than 2 mg.

When the excipient used is an approximately 85-15 PLGA with a viscosity comprised between 1.2 and 1.6 dl/g in HFIP, the microimplant according to the invention can have, for example, a diameter of 0.8 to 0.9 mm and comprise approximately 36% by weight of triptorelin salt and approximately 64% by weight of approximately 85-15 PLGA. For a release period of approximately 4 months, such a microimplant can in particular contain approximately 2.16 mg of triptorelin acetate and approximately 3.84 mg of approximately 85-15 PLGA.

A person skilled in the art can of course choose to use other lactic acid and/or glycolic acid polymers or copolymers or also a mixture of lactic acid and/or glycolic acid polymers and/or copolymers, or to have other proportions of triptorelin salt and PLGA; in this case, the molecular mass of the PLGA and the weight of the microimplants will be adapted in order to obtain the desired effect.

A subject of the invention is therefore also a method for treating a patient needing regular administration of an LHRH analogue, said method consisting of the injection and implantation in this patient of a solid sustained-release formulation according to the invention, either at the monthly doses usually used for said LHRH analogue, or at lower doses made possible by the formulation and its administration profile.

According to the size of the formulation obtained, the attending doctor or vet can use injection devices such as those described in the PCT Application WO 98/24504 or syringes of standard size in order to carry out the administration.

The term “approximately” refers to an interval around the value considered. As used in the present Application, “approximately X” signifies an interval of X minus 10% of X to X plus 10% of X, and preferably an interval of X minus 5% of X to X plus 5% of X. If it is a question more specifically of temperature intervals, “approximately Y° C.” then refers to an interval of Y minus 10° C. to Y plus 10° C., and preferably an interval of Y minus 5° C. to Y plus 5° C.

Unless otherwise specified, all the technical and scientific terms used here have the same meaning as that commonly understood by an ordinary specialist in the field to which this invention belongs. Similarly, all the publications, patent applications, all patents and all other references mentioned here are incorporated by way of reference.

The following examples are presented in order to illustrate the above procedures and should in no case be considered as a limit to the scope of the invention.

### EXAMPLES

### General Procedure for Preparation of Formulations of the Examples

The polymer or copolymer on the one hand and the triptorelin acetate on the other hand are weighed, then their powders are mixed using a Turbula T2C INS4586 device (speed of rotation 42 rpm) and transformed (by compression or compaction) to granules the size of which does not exceed 1.4 or 1.5 mm (control ensured by sieving). The water content present in the granules is determined in a sample then adjusted to the desired level by drying under vacuum at ambient temperature. The dried granules are then subjected to a melting-extrusion process at a speed of 10 rpm (Scamex 8/12 mm extruder (Scamia)) whilst the temperature during this process is maintained at the desired temperature (for the particular case of the implants comprising at least 50% triptorelin acetate). Two devices are used to carry out the melting-extrusion; their characteristics are shown in the table below.

After chemical analysis, the extrudate is cut manually into implants which are then γ-irradiated (25 kGy). The implants are then ready to be loaded into injection devices.

### Example 1

An implant of 5.9 mg, measuring 0.85 mm in diameter and approximately 28 mm in length and comprising 36% by weight of triptorelin acetate (purity ≧98.5%) and 64% by weight of 85:15 PLGA (Boehringer Ingelheim; viscosity index VI in hexafluoroisopropanol: 1.2 dl/g≦VI≦1.6 dl/g), is produced according to the general procedure described above.

### Example 2

An implant of 6.01 mg, measuring 0.85 mm in diameter and approximately 27 mm in length and comprising 36% by weight of triptorelin acetate (purity ≧97.5%) and 64% by weight of 85:15 PLGA (Boehringer Ingelheim; viscosity index VI in hexafluoroisopropanol: 1.2 dl/g≦VI≦1.6 dl/g), is produced according to the general procedure described above.

### Example 3

An implant of 7.5 mg, measuring 0.85 mm in diameter and approximately 25 mm in length and comprising 50% by weight of triptorelin acetate (purity ≧98.5%) and 50% by weight of 85:15 PLGA (Boehringer Ingelheim; viscosity index VI in hexafluoroisopropanol: 1.2 dl/g≦VI≦1.6 dl/g), is produced according to the general procedure described above.

### Example 4

An implant of 16.2 mg, measuring 0.85 mm in diameter and approximately 20 mm in length and comprising 56% by weight of triptorelin acetate (purity ≧98.5%) and 50% by weight of 75:25 PLGA (viscosity index VI in hexafluoroisopropanol: VI=0.95 dl/g), is produced according to the general procedure described above. During the melting-extrusion, the temperature is maintained at 144-147° C.

### Example 5

An implant of 9.1 mg, measuring 0.85 mm in diameter and approximately 22 mm in length and comprising 65% by weight of triptorelin acetate (purity ≧97.5%) and 35% by weight of 75:25 PLGA (viscosity index VI in hexafluoroisopropanol: VI=0.95 dl1 g), is produced according to the general procedure described above. During the melting-extrusion, the temperature is maintained at 144-147° C.

Pharmacokinetic Properties of the Formulations of the Invention

Implants according to Example 4 were administered by intra-muscular route on the one hand to Beagle dogs (weight approximately 12 kg) into a muscle of the rear paw and on the other hand to humans. In the dogs, a plasma assay revealed that the level of triptorelin remained constantly above 0.1 ng/ml over a period of more than 80 days whereas the level of testosterone measured was constantly below the castration level (0.24 ng/l) between the 21^{st} and the 113^{th} day after administration of the implant; in the humans, the triptorelin level remained constantly above 0.03 ng/ml over a period of more than 112 days whereas the level of testosterone measured was constantly below the castration level (0.50 ng/l) between the 15^{th} and the 105^{th} day after administration of the implant.

1. Solid sustained-release formulation for parenteral administration comprising:
a) triptorelin acetate, and
b) one or more excipient(s) comprising a lactic acid and/or glycolic acid polymer or copolymer or a mixture of lactic acid and/or glycolic acid polymers and/or copolymers, said formulation including 10 to 99% triptorelin acetate by weight relative to the total weight of the formulation, wherein said formulation is obtained by a process comprising the melting of the mixture of triptorelin acetate and excipient or excipients during the melting-extrusion of triptorelin acetate with the excipient or excipients, and wherein
said formulation releases the triptorelin acetate over a period of at least one week once administered to a patient by parenteral route.

2. Sustained-release formulation according to claim 1, wherein said formulation releases the triptorelin acetate in an effective dose for a period of at least 14 days.

3. Sustained-release formulation according to claim 1, wherein said formulation releases the triptorelin acetate in an effective dose for a period of at least 28 days.

4. Sustained-release formulation according to claim 1, wherein said formulation releases the triptorelin acetate in an effective dose for a period of at least 90 days.

5. Sustained-release formulation according to claim 1, wherein said formulation comprises 20 to 90% by weight of triptorelin acetate relative to the total weight of the formulation.

6. Sustained-release formulation according to claim 1, wherein said formulation comprises 30 to 70% by weight of triptorelin acetate relative to the total weight of the formulation.

7. Sustained-release formulation according to claim 1, wherein said lactic acid and/or glycolic acid polymer or copolymer or the mixture of lactic acid and/or glycolic acid polymers and/or copolymers is a lactic acid and glycolic acid copolymer or a mixture of such copolymers.

8. Sustained-release formulation according to claim 7, wherein said lactic acid and glycolic acid copolymer or copolymers are copolymers comprising 50% to 85% units derived from lactic acid and 15% to 50% units derived from glycolic acid.

9. Sustained-release formulation according claim 1, wherein said formulation comprises more than 35% triptorelin acetate by weight relative to the total weight of the formulation and wherein the mixture of triptorelin acetate with the polymer or copolymer excipient or excipients has been dried before the melting-extrusion so that its water content does not exceed 8% by weight.

10. Sustained-release formulation according to claim 9, wherein the mixture of triptorelin acetate with the polymer or copolymer excipient or excipients has been dried before the melting-extrusion so that its water content does not exceed 4% by weight.

11. A solid sustained-release formulation for parenteral administration comprising a melt-extruded mixture of:
a) triptorelin acetate, and
b) one or more excipient(s) comprising a lactic acid and/or glycolic acid polymer or copolymer or a mixture of lactic acid and/or glycolic acid polymers and/or copolymers,
said formulation including 10 to 99% triptorelin acetate by weight relative to the total weight of the formulation, and wherein said formulation exhibits at least one of: a sustained release of an effective dose of triptorelin acetate for a period of at least one week or a reduction in release burst of triptorelin acetate compared to standard formulations when administered to a patient by parenteral route.
